(19) United States
(12) Patent Application Publication
(10) Pub. No.: US 2015/0017669 A1
(43) Pub. Date: Jan. 15, 2015

(54) PROCESS AND ITS APPLICATION FOR IMPROVING REPRODUCIBILITY IN MALDI-TOF GLYCAN PROFILING OF HUMAN SERUM: EXPERIMENTAL PROCEDURE AND APPLICATION TO THE SCREENING FOR OVARIAN TUMORS

(71) Applicant: HUDSON SURFACE TECHNOLOGY, INC., OLD TAPPAN, NJ (US)

(72) Inventors: Yohahn Jo, West New York, NJ (US); Yangsun Kim, Gyeonggi-do (KR)

(21) Appl. No.: 13/938,751

(22) Filed: Jul. 10, 2013

(51) Int. Cl. G01N 33/66

(52) U.S. Cl. CPC G01N 33/66; G01N 2400/00; G01N 2800/36
USPC 435/18

(57) ABSTRACT

Disclosed is that the most crucial point in the quantitative analysis of MALDI-TOF MS is to minimize quantitative variances (RSDs, relative standard deviations) of peak intensities of obtained spectrum data. The minimized RSD is translated into improved reproducibility of the data and finally leads to high accuracy of the results. Several technical procedures based on the concept of automatization to reduce the RSDs (to less than 10%) in the MALDI-TOF MS of serum glycans were proposed for the preparation steps of extracting the targeted glycans from human blood and sample loading processes onto the plates. The presented techniques described in the examples proved the contributions of the present invention to enhanced reproducibility of the MALDI-TOF MS and consequently to improved screening accuracy for the differentiation of the benign ovarian tumor patients from the borderline ovarian tumor patients in comparison with accuracy of differentiation based on other analysis methods.

### BACKGROUND OF THE INVENTION

(a) Field of the Invention

The present invention relates to a method for glycan analysis with improved reproducibility of the quantitative MALDI-TOF mass spectrometric profiling of the glycans in human serum. Further, the present invention relates to a cancer screening method by using the glycan analytical method. In addition, the present invention relates to a differentiation method of benign ovarian tumors from borderline ovarian tumors by using the glycan analytical method.

(b) Description of the Related Art

Recently, studies that use serum glycans for identifying and predicting specific diseases, thereby allowing clinical diagnosis, research studies, and practical applications, have been well received. From previous studies and patents, it has been discovered that the glycosylation of tumors can change for the cases of ovarian, breast, prostate, liver, lung, gastric, pancreatic, and many other malignant tumors.

It has been reported that glycosylation of proteins changes when tumors arise, which alters the levels of oligosaccharides in serum glycoproteins. Some of the glycoprotein changes are suggested as potential biomarkers for various diseases. Particularly, a considerable part of mucin, which is classified as one of highly glycosylated glycol-proteins, is known as a glycan. For instance, U.S. Pat. No. 7,651,847 demonstrated that screening and diagnosing ovarian and breast cancers and predicting the cancer development stages were possible by following the pattern analysis of the mass spectra of oligosaccharides isolated and purified from blood samples of cancer-inflicted patients.

Most of the cancer screening and diagnostic methods of quantitative measuring tumor markers in human blood have been attempted by a technique which finds the value of a single marker in the human serum by immune response of an antigen-antibody and then compares it with cut-off values derived from a clinical cumulative database. For example, CA-125 (cancer antigen 125, mucin 16) is reported as a common biomarker for ovarian cancer; the amount of CA-125 produced in the body increases for patients inflicted with ovarian cancer. However, CA-125 is a highly heterogeneous glycoprotein and this makes the antigen-antibody immunological response of the mucin inaccurate and difficult to quantify depending on the various types of ovarian cancer. Women with endometriosis or pelvic inflammatory disease also display high concentrations of CA-125, which causes a tendency of false positive responses. Consequently, CA-125 is not a proper cancer marker to be selected for diagnosing the beginning stage of ovarian cancer.

In order to overcome the limit of a single tumor marker such as CA-125, in the US patent mentioned above, all glycans in the serum of a cancer patient were separated and quantitatively measured through mass spectrometry. The profiles of the glycan spectrum were compared by pattern analysis with those of glycans from the serum of people who are not cancer patients. The patent has proved the successful screening results of the early stage of ovarian and breast cancer patients from healthy persons.

The Mass spectrometry they used was MALDI-FTICR (Matrix Assisted Laser Desorption and Ionization—Fourier Transform Ion Cyclotron Resonance), which has excellent power at high resolution. However, it is clear that that instrument is not appropriate for local clinics or diagnostic laboratories to use for early stage cancer screening purposes due to the expensive price of the instrument and the long time consumed to obtain the mass spectrometry. To conquer these handicaps, the present invention adapts the MALDI-TOF (Matrix Assisted Laser Desorption and Ionization—Time of Flight), which is one of the most widely used instruments, and provided mass spectrometry and analyzes hundreds of glycan samples quickly at one time of sample loading, resultantly offering the technology to discover multiple cancer markers and to screen cancer and tumor patients from healthy persons by using it.

In case of separating early stage cancer patients from healthy persons, sometimes the differences of the markers' intensities are not distinctive enough to screen, so the reproducibility of the MALDI-TOF mass spectrometric signals must be very critical, for MALDI-TOF tends to show the variation of peak intensity intrinsically from each data gaining time. To drastically improve the reproducibility of MALDI-TOF mass spectrometric signals, the present invention introduces new processes for the glycan extraction step and the glycan loading step onto the plate for instrumental measurement.

### SUMMARY OF THE INVENTION

The present invention relates to an analytical method of the glycans and its application to improve reproducibility of quantitative MALDI-TOF mass spectrometric profiling of the glycans in human serum. When we want to differentiate and screen out the patients from healthy peoples only by MAlDI-TOF mass spectrometric quantitative analysis of the glycans extracted from human serum, and especially when the intensity variations of the marker peaks in the spectra of the glycans between patients and healthy peoples are not distinctive enough due to the early stage of the diseases, then the reproducibility of the MALDI-TOF mass spectrometric analysis must be established. Therefore, it is very important for the coefficient of variation of each peak's intensity at every measurement of MALDI-TOF to be minimized and consistent.

For example, when patients of early stage ovarian cancer or breast cancer are screened out from healthy persons, or when patients of borderline ovarian tumors (possibly developed as ovarian cancers afterwards) are separated from patients of benign ovarian tumors, in which the MALDI-TOF mass spectrometric patterns of glycans for both cases are quite similar, the accuracies of the results of quantitative analyses of the multiple markers and their reproducibilities are crucial for screening and separation of the corresponding diseases.

The present invention provides methods to minimize the causes to affect the reproducibilities of peak intensities of the glycan as the quantitative analysis of MALDI-TOF mass spectrometry, resulting in maximizing the quantitative data reproducibility by way of introducing several new processes of sample preparation step for glycan extraction and of a glycan loading step on the plate for MALDI-TOF measurement. In addition, the present invention not only offers the method to analyze multiple numbers of glycans in different human sera by MALDI-TOF mass spectrometry concurrently as one batch, but also maximizes the reproducibilities of the above data at the same time.

In one aspect, the present invention provides a method of glycan analysis from the human serum including the steps of:

(a) injecting individual human serum samples into each area of a Plate #1 having a plurality of sample-loading areas;

(b) extracting at least one glycan from the sample by introducing glycan-releasing enzyme into each area of the Plate #1;

(c) preparing a Plate #2 having a plurality of sample-loading areas by spreading a matrix solution for MALDI-TOF mass spectrometry at least two times onto each area of the Plate #2 at 40 to 65° C., and subsequently drying and crystallizing the matrix;

(d) loading a solution including at least one glycan extracted from the step (b) onto each area of the Plate #2;

(e) drying the Plate #2 under reduced pressure; and

(f) obtaining a mass spectrum of at least one glycan from each area in the Plate #2 using MALDI-TOF,

wherein an average coefficient of variation of intensity of each obtained peak from the mass spectrometry is less than 10%.

Preferably, the number of sample-loading areas of the Plate #2 may be equal to or more than the number of sample-loading areas of the Plate #1.

Further preferably, the Plate #1 may be a plate having multiple wells of a number of 6, 12, 24, 48, 96, or 384.

Further preferably, the Plate #2 may be a sample microfocusing plate for MALDI-TOF mass spectrometry, including at least two sample-loading areas including a central portion with a hydrophilic surface for condensation of a loaded material and a peripheral portion surrounding the central portion with a hydrophobic surface.

Further preferably, the step (b) may include the following steps of:

(b-1) denaturing proteins in the human serum sample by heating the Plate #1;

(b-2) treating the glycan-releasing enzyme with the sample to release glycans from the proteins in the sample; and

(b-3) separating at least one glycan from the proteins in the sample.

Also, as a good example, the above step (b-1) may be performed by heating the Plate #1 after adding dithiothreitol, 2(β)-mercaptoethanol, or TCEP [tris(2-carboxyethyl)phosphine] into the sample to denature the proteins in the sample.

Further preferably, the step (b-2) may be performed in a water bath including a temperature controller, a microwave source radiating waves horizontally, and a system for generation of air bubbles.

Further preferably, the step (b-2) may be performed in the water bath where the sample is irradiated by microwaves directed horizontally in parallel to the surface of the water, while a reaction-containing lower portion of said Plate #1 is submerged in the water bath.

Further preferably, the glycan-releasing enzyme in the step (b) may be a peptide-N-glycosidase F.

Further preferably, the matrix in the step (c) may be 2,5-dihydroxybenzoic acid.

Further preferably, the Plate #2 in the step (c) may be prepared by placing the Plate #2 on top of a heating plate at a temperature between 40 and 65° C. and then spreading the matrix solution at least two times onto each area of the Plate #2, and subsequently drying and crystallizing the matrix.

Further preferably, the step (e) may be performed under reduced pressure of 6˜8×10⁻² torr.

Further preferably, the glycan may be the one selected from the group consisting of mannose, pentose, hexose, N-acetylglucosamine, N-acetylhexhoamine, N-acetylneuraminic acid, hexosamine, sialic acid including N-acetylneuraminic acid and N-glycolylneuraminic acid, uronic acid including GlaA, and a combination thereof.

Further preferably, each human serum sample in the step (a) may be from a plurality of cancer patients. In particular, the step (a) may include the treatment of each serum of multiple cancer patients at each well of the Plate #1 having multiple wells.

Further preferably, the each human serum sample in the step (a) may be from at least one healthy person and at least one cancer patient. In particular, the step (a) may include the treatment of each serum of multiple healthy persons and each serum of multiple cancer patients at each well of the Plate #1 having multiple wells. In this case, the above analytical method for glycan may further including a step (g) of selecting tumor-specific glycan by comparing each mass spectrum of the glycan extracted from each area in the Plate #2.

Further preferably, each human serum sample in the step (a) may be from at least one benign ovarian tumor patient and at least one borderline ovarian tumor patient. In particular, the step (a) can include the treatment of each serum of multiple benign ovarian tumor patients and each serum of multiple borderline ovarian tumor patients at each well of the Plate #1 having multiple wells. In this case, the above analytical method for glycan may further include the step (g) of selecting a tumor-specific glycan by comparing each mass spectrum of the glycan extracted from each area in the Plate #2.

In another aspect, the present invention provides a cancer screening method including the steps of:

(a) injecting each serum sample from healthy people and cancer patients into each area of the Plate #1 having a plurality of sample-loading areas;

(b) extracting at least one glycan from the sample by introducing glycan-releasing enzyme into each area of the Plate #1;

(c) preparing the Plate #2 having a plurality of sample-loading areas by spreading a matrix solution for MALDI-TOF mass spectrometry at least two times onto each area of the Plate #2 at 40 to 65° C., and subsequently drying and crystallizing the matrix;

(d) loading a solution including at least one glycan extracted from the step (b) onto each area of the Plate #2;

(e) drying the Plate #2 under reduced pressure; and

(f) obtaining a mass spectrum of at least one glycan from each area in the Plate #2 using MALDI-TOF.

In another aspect, the present invention provides a differentiation method of benign ovarian tumors from the borderline ovarian tumors, including:

(a) injecting each serum sample from benign ovarian tumor patient and borderline ovarian tumor patient into each area of a Plate #1 having a plurality of sample-loading areas;

(b) extracting at least one glycan from the sample by introducing a glycan-releasing enzyme into each area of the Plate #1;

(c) preparing a Plate #2 having a plurality of sample-loading areas by spreading a matrix solution for MALDI-TOF mass spectrometry at least two times onto each area of the Plate #2 at 40 to 65° C., and subsequently drying and crystallizing the matrix;

(d) loading a solution including at least one glycan extracted from the step (b) onto each area of the Plate #2;

(e) drying the Plate #2 under reduced pressure; and

(f) obtaining a mass spectrum of at least one glycan from each area in the Plate #2 using MALDI-TOF.

### BRIEF DESCRIPTION OF THE DRAWINGS

FIG. 1 shows the sequential steps to differentiate benign ovarian tumor patients from borderline ovarian tumor patients by using the glycan analysis method offered by one example of the present invention.

FIG. 2 shows the sequential steps to extract one or more glycans from human blood in the process of the glycan analysis method according to one example offered by the present invention.

FIG. 3a shows how to load the Plate #1 on the surface of water bath irradiating microwaves in the process of the glycan analysis method according to the example offered by the present invention.

FIG. 3b shows the schematic diagram of the Plate #1 composed of 96 wells as viewed from above. In this diagram, each well is filled with serum, and the proteins are denaturated, wherein they are enzymatically digested, which releases glycans from the proteins. Each well acts as a reactor.

FIG. 4a shows the schematic cross-sectional diagram of the water bath having the Plate #1 horizontally placed on the surface of the water, while irradiating microwaves from the peripheral of the water bath to the center. Compressed air from the outside is designed to flow through holes located in a circle at the bottom, which forms bubbles and turbulence from the bottom to the surface, resulting in a cooling effect.

FIG. 4b shows a graph tracing the reaction temperature (° C.) of the water bath and electric current (A) which irradiates the microwaves for a one hour reaction time from the start.

FIG. 5 shows the usage of the Plate #2 for the MALDI-TOF analysis of glycans as one example of the present invention. Each area of the Plate #2 can be covered by a mixed solution of matrix and glycans, or the area can be evenly covered by the matrix first before the addition of glycans.

FIG. 6a shows a mass spectrum of the glycans finally eluted from a 10% ACN solution according to the glycan analytical method of one example, originated from human sera of Sigma Aldrich.

FIG. 6b shows a mass spectrum of the glycans finally eluted from a 20% ACN solution according to the glycan analytical method of one example, originated from human sera of Sigma Aldrich.

FIG. 7a shows one mass spectrum as a representative graph of the glycans finally eluted from a 10% ACN solution according to the glycan analytical method of one example, originated from a borderline ovarian tumor patient.

FIG. 7b shows one mass spectrum as a representative graph of the glycans finally eluted from a 10% ACN solution according to the glycan analytical method of one example, originated from a benign ovarian tumor patient.

FIGS. 8a and 8b show bar graphs of average, standard deviation, and coefficient of variation values of the absolute intensities measured from the mass spectra of the same 32 samples. Four measurements of each sample were made from different areas, and all the intensities were obtained at a 1485.53 mass-to-charge ratio.

FIG. 8a shows the MALDI-TOF spectrum obtained from the mixed crystals of the glycans and the matrix which were pre-mixed and then deposited on the circle of regular plate having no self-focusing function and then dried at ambient conditions.

FIG. 8b, on the contrary, shows the MALDI-TOF spectrum where the glycans' solution was deposited on the matrix layer which was made in advance on the circle of the self-focusing plate, and then quickly dried under reduced pressure.

FIG. 9 shows schematized steps and the time consumed starting from the preparation stage of 96 blood samples of 96 patients using 96 wells of Plate #1 as one batch, the sample loading stage using Plate #2, the mass spectrum obtaining stage, and the interpretation/finalizing stage of benign ovarian tumor or borderline ovarian tumor, according to the method of the present invention.

### DETAILED DESCRIPTION OF THE PREFERRED EMBODIMENTS

In the present invention, quantitative analysis of glycans existing in human serum was realized by MALDI-TOF mass spectrometry.

In a specific embodiment of the present invention, an enzyme digestion reaction to obtain the glycans could be completed within 10 minutes by microwave irradiation accompanied by introducing an air cooling effect by adopting bubble creating design, resulting in maximizing the effect of microwave irradiation while maintaining the exact temperature of the reaction.

In addition, MALDI-TOF analysis was executed with the Plate #2, having self-focusing multiple areas arrayed as the pattern #2. Before loading the samples onto the circular area of the Plate #2, however, the matrix crystal was layered at each area by spraying the matrix-diluted solution several times and by heating the plate while spraying to induce fast drying, consequently making the matrix crystal more compact and even. After this, the solution of glycans extracted from the serum sample was deposited on the matrix pre-spotted area of the Plate #2 and then it was quickly dried at reduced pressure in order to induce co-crystallization of the matrix and the glycan as evenly as possible.

By introducing a series of new processes into the analytical method of serum glycans by using MALDI-TOF mass spectrometry, the coefficients of variation of the glycan intensities of the different spectra at the same molecular weight (which is m/z) decreased to less than 10%, or more preferably to the range of 5%, while the CVs of the glycans measured by conventional technology was around 20%. This enhanced reproducibility of the new MALDI-TOF analytical methods can not only screen out the cancer patients such as those with ovarian cancer, which are known to be difficult to screen at an early stage, can but also differentiate tumor patients whose physiological characteristics are quite similar.

According to specific examples of the invention, Group #1 of mass spectra were obtained from the sera of 169 benign ovarian tumor patients by applying the quantitative mass spectrometric analytical method presented by the present invention. Then, these mass spectra of Group #1 were compared with the mass spectra of Group #2 whose glycans were derived from the sera of 68 borderline ovarian tumor patients, and then 72 tumor-specific peaks of m/z, which showed up at every mass spectrum (in other words, having 100% frequencies), were selected as markers. By comparing the analysis of these markers, the cut-off values of each individual marker were fixed.

In addition, by the two separate blind validation tests, the glycan mass spectrometry analysis has proved to have 23% better sensitivity and 13% better specificity than the conventional tumor marker of CA-125, respectively. Furthermore, by using only one MALDI-TOF instrument, the sera of about 100 people can be concurrently analyzed and then interpreted for the screening of cancer and/or for differentiating tumors within 48 hours, which offers commercially available processes.

Therefore, the present invention provides analytical methods of the glycans to improve the reproducibility of quantitative MALDI-TOF mass spectrometric glycan profilings of human serum and also offers the screening method for cancer by using these methods.

In a specific embodiment of the present invention, the analytical method of glycans from samples is presented in the following steps. It should be noted that the average coefficients of variation of the intensity of each peak obtained in the whole spectra of this method is less than 10%:

(a) injecting each human serum sample into each area of the Plate #1 having a plurality of sample-loading areas;

(b) extracting at least one glycan from the sample by introducing glycan-releasing enzyme into each area of the Plate #1;

(c) preparing the Plate #2 having a plurality of sample-loading areas by spreading a matrix solution for MALDI-TOF mass spectrometry at least two times onto each area of the Plate #2 at 40 to 65° C., and subsequently drying and crystallizing the matrix;

(d) loading a solution including at least one glycan extracted from the step (b) onto each area of the Plate #2;

(e) drying the Plate #2 under reduced pressure; and

(f) obtaining a mass spectrum of at least one glycan from each area in the Plate #2 using MALDI-TOF.

Hereinafter, the present invention is described in more detail in a step-by-step manner.

In step (a), the serum samples are placed in each well of the Plate #1 which consists of multiple wells.

This Plate #1 in the present invention can play a role of a micro-reactor to pre-treat the samples for MALDI-TOF mass spectrometric analysis. Enzyme digestion reactions may be performed and then glycans may be released from the proteins at each well of the Plate #1. This Plate #1 usually consists of multiple areas (or wells), and these areas may respectively hold tubes carrying samples or these wells may contain samples directly in them without the separate tubes, which results in the rapid analysis of many samples at one time.

The numbers and the shapes of the wells in the above Plate #1 are not specifically limited, but preferably, two or more wells can be arrayed in the Plate #1. For example, commercially available plates with 6, 12, 24, 48, or 96 wells can be used.

In the present invention, each serum sample from each patient can be injected into each well of the Plate #1. Here, the injection means to put the sample into the well of the Plate #1 directly or to place the sample-containing tube in the holes (areas) of the Plate #1. The samples can be injected as a mixed solution with a buffer solution after blending prior to the injection. The buffer solution can be an ammonium bicarbonate solution, but other buffer solutions can be used.

The step (b) is the glycan extraction step by enzyme digestion reaction at each area of the Plate #1 to extract one or more glycans from the samples.

The above step (b) can be further divided into following (b-1) to (b-3):

(b-1) denaturing the proteins in the human serum sample by heating the Plate #1;

(b-2) treating glycan-releasing enzyme with the sample to release glycans from the proteins in the sample; and

(b-3) separating at least one glycan from the proteins in the sample.

The above step (b-1) is the denaturing reaction of the proteins. The sample in the Plate #1 can be mixed with dithiothreitol and heated in the water bath at temperatures above 90° C., or at 95° C. as a better example, in order to denature the proteins.

The above step (b-2) is the reaction step of the sample with the enzyme. As an example, peptide N-glycosidase F (PNGase F) can be injected into the sample to extract the glycans.

To get a good result, the above enzyme digestion reaction can be executed in a water bath while irradiating the bath with microwaves horizontally parallel to the surface of the water bath.

Enzyme reactions are usually executed in the water bath for 16 to 24 hours (so-called overnight) at 37° C. In the present invention, however, the enzyme reaction could be rapidly completed by irradiating the enzyme reaction with microwaves horizontally to the surface of the water by floating or placing the Plate #1 which contains a large number of sample-containing wells in order to process many samples simultaneously in a batch for the purpose of commercial application of the present invention. As an example, the water bath harboring the Plate #1 can be irradiated with microwaves of 400 W for 10 minutes at 37° C. Once the samples are exposed to microwaves, microwave energy stimulates the movement of all molecules including serum proteins in each well of the plate, which increases the reaction speed, quickly reaching the ending point of the enzyme digestion reaction.

Preferably, the above water bath can include not only a magnetron, which is the source of microwaves, but also inlet holes for the air bubbles. Since the microwaves are controlled by a temperature-dependent device, a higher temperature induced by microwaves in the water bath will stop the generation of microwaves above a certain set temperature. In order to maintain the microwave generation and thus to further accelerate the enzyme reaction with microwaves, the water bath should be cooled to below the set temperature immediately after reaching the set temperature. Air bubbles which are generated from holes at the bottom of the water bath equipped with a small air pump will induce the flow of air bubbles from the bottom for fast heat dissipation, which leads to a lower temperature and consequent switching on of the microwave generation. In this mechanism, the temperature of the water bath is maintained with oscillations in a very small range (±0.5° C.), while microwaves are emitted constantly at regular time intervals. This constant microwave irradiation eventually results in the completion of the enzyme reaction in a much shorter time, emphasizing the need for a cooling device for the water bath such as the example of air bubbles and related air pumps in this embodiment.

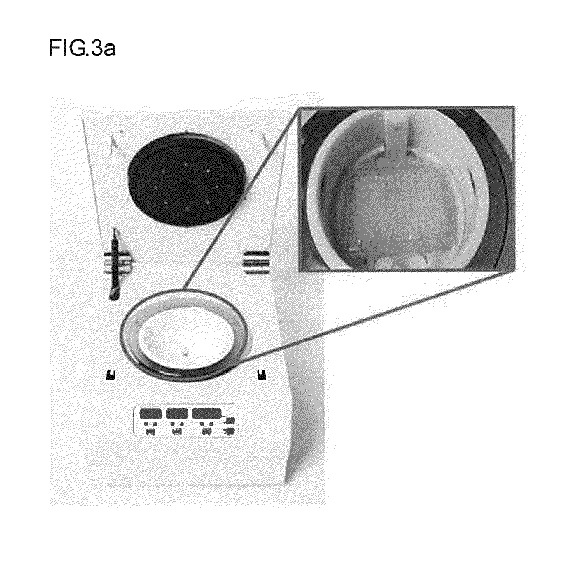

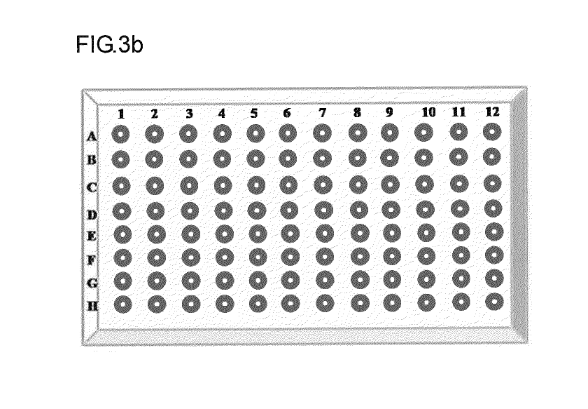

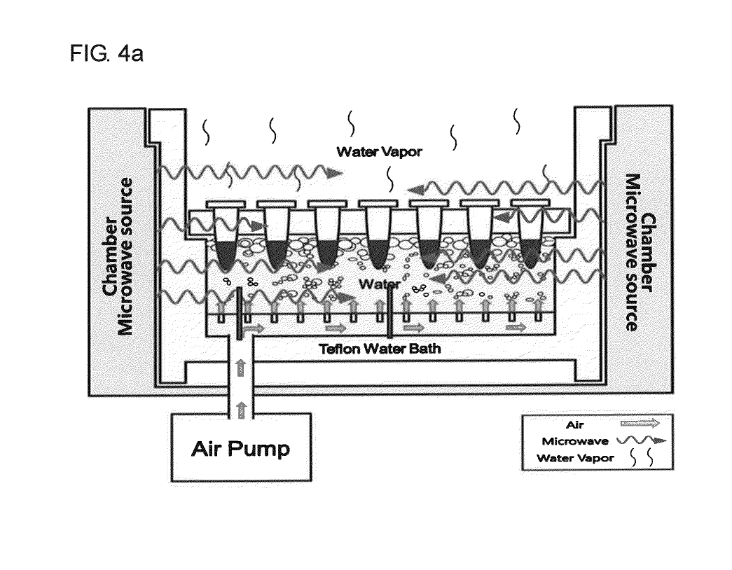

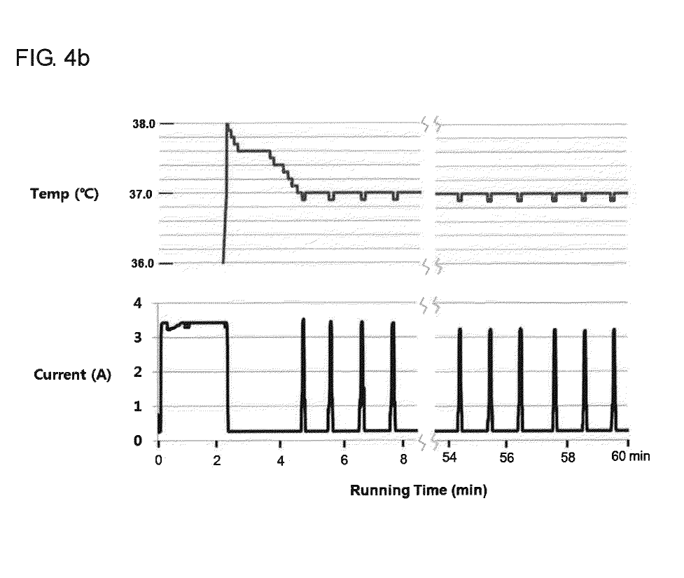

FIG. 3a shows the loading process of the Plate #1 on the surface of the water bath in the microwave irradiation system, and FIG. 4a is a schematic diagram of the water bath equipped with the microwave irradiating system, which shows a conceptual side view of the bath. As shown in above figures, the Plate #1 can be loaded parallel on the surface of the water bath and the microwaves are radiated horizontally from the side wall of the cylindrical bath, while air bubbles travel continuously from the entrance holes at the bottom to the surface by forming the bubbles and turbulence. The above schematic design is, however, just one example to embody the present invention, which means that the invention is not confined to this example and this equipment at all.

The above step (b-3) is the separation step of one or more glycans from serum proteins in the samples. As one example, the proteins can be removed by alcohol precipitation after enzyme reaction.

More particularly, the glycans can be separated by the step of precipitating the above proteins by adding absolute ethanol to the sample solution after the enzyme reaction, the step of removing the precipitated proteins, and the step of eluting one or more glycans from the above step.

As an example, after mixing the sample solution with ethanol in a 1:4 (volume/volume) ratio, the proteins can be precipitated at −80° C. Once the precipitates are formed, they can be removed by centrifugation, and the remaining supernatant solutions are transferred to new tubes, where all solvents are removed in speed vacuum dryers. From the completely dried materials, one or more glycans can be purified and eventually eluted by the solid phase extraction process.

The step to elute one or more glycans from the above sample can be carried out by pouring an acetonitrile solution into the sample. For example, a graphite carbon column can be prepared by equilibration with distilled water, with an acetonitrile solution, and with distilled water one more times, sequentially. The samples dried by the speed-vacuum evaporator are then dissolved in distilled water, and the solution is introduced into the column.

As the next step, distilled water is used to wash off any salt from the columns, and secondly, an ACN solution is injected to finally elute glycans. The ACN solution for eluting glycans can have a 10% and 20% concentration in distilled water, respectively, or a 40% concentration in water additionally including trifluoroacetic acid at a 0.05% concentration. The volume of the above solutions for the elution of one or more glycans can be around 4 mL to 10 mL.

The above step (a) and step (b) in the present invention are the process for treating the samples. Therefore, a multi-pipette system can be used to inject several samples into the multiple wells or to extract several samples in the Plate #1 at the same time. As an example, Liquidator 96 provided by Mettler Toledo can be used in this step, but is not limited to this.

In the step (c), the matrix solution is sprayed onto the area and the matrix on each area of the Plate #2 having multiple areas is crystallized. The Plate #2 plays a role of a sample-loading plate to place each extracted glycans for the MALDI-TOF mass spectrometry. The pre-deposited area of the above Plate #2 can be an array of spots such as circles or squares, which are shaped by grooves for holding the sample or matrix within the designated spots. The plate can be made of stainless steel, and the shapes of the area are not limited to certain figures. The areas in the Plate #2 can be arrayed with a regular pattern, which can handle many different samples at the same time. The number of areas in the Plate #2 can be the same number of areas in the Plate #1, or can be different. As a good example of the Plate #2, the number of areas can be more or the same number as the areas in the Plate #1.

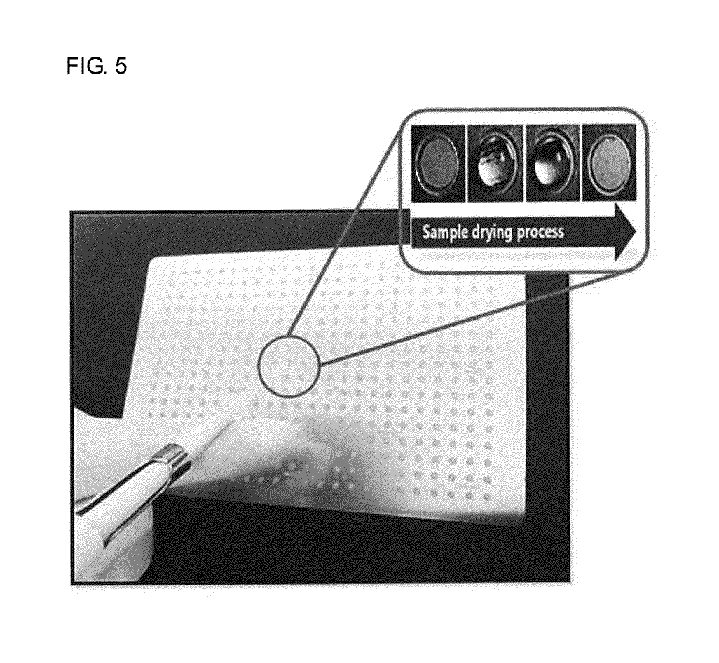

As one example, the Plate #1 can be a plate having 96 wells, and the Plate #2 can have 384 individual areas for the loading of glycans and matrix. FIG. 5 shows the moment of depositing a solution of the sample onto one spot of the Plate #2 which has 384 spots in an embodiment. However, the actual number of sample loading areas on the Plate #2 could be different from 384 of the above example, and could also be identical to or smaller than the number of wells on the Plate #1.

The surface of the Plate #2 can be metal such as aluminum, stainless steel, a copper alloy, galvanized iron, a gold film, or a silver film. But the plate is not limited only to these materials.

As a good example, the Plate #2 can be a so-called micro-focus self-condensing plate whose surface was already treated with some special chemicals for inducing highly condensed deposition of the sample solution onto each spot owing to hydrophilic-hydrophobic repulsive surface tension. In addition, the plate can be a pre-spotted micro-focus plate of which self-focusing spots are already coated with a matrix such as 2,5-dihydroxybenzoic acid (generally called DHB) in order to analyze the glycans more effectively. U.S. Pat. No. 7,619,215 explains the invention of the micro-focus plate in detail.

In another aspect, the present invention is characterized by using the plate which has each spot pre-coated with the matrix material for MALDI-TOF mass spectrometry before applying the sample solution onto the spot. This pre-spotting of the matrix prior to the loading of glycan samples maximizes the sensitivity and reproducibility of MALDI-TOF mass spectrometry. Therefore, the Plate #2 in the present invention can be provided as a plate of which areas for the glycan sample are already coated with the matrix by a spraying machine before loading the glycan solution.

In a more desirable embodiment, the coating of sample-loading areas with the matrix on the Plate #2 can be performed on top of a heating plate at temperatures between 40° C. and 55° C. for rapid and uniform crystallization of the matrix. This coating process is repeated several times to deposit at least a certain amount of matrix on each area.

In an exemplary embodiment, a matrix in an ethanol solution can be sprayed two times or up to 10 times, in order to spread the matrix more evenly to each sample-loading spot of the Plate #2. In a desirable embodiment, a sample-loading spot on the Plate #2 is sprayed with the matrix solution 4 to 8 times. When the matrix solution is sprayed on the plate, the plate can be heated at around 40˜55° C. so that the ethanol solvent in the matrix solution is quickly evaporated from the spot, leading to uniform and dense crystallization of the matrix due to fast drying.

The step (d) includes the loading of solutions containing one or more glycans extracted from the above step (b) onto the areas of the Plate #2 which were already covered with uniform matrix crystals as described in the previous section.

One or more glycan(s) as the outcome of the preparation step using the Plate #1 can be injected in a solution state onto one or more spots of the Plate #2, where the matrix crystals for MALDI-TOF mass spectrometry are pre-spotted. The glycans eluted in the 10% and 20% ACN solutions are neutral glycans, which are not easily ionized during the MALDI-TOF analysis process. Therefore, NaCl can be added to induce the ionization of the neutral glycans for the MALDI-TOF mass spectrometry in a positive mode. On the other hand, the glycans eluted in the 40% ACN after the solid phase extraction are acidic glycans, which can be analyzed in the negative mode of MALDI-TOF mass spectrometry without NaCl.

In an exemplary embodiment, the glycan solution can be reconstituted by introducing pure water (Nanopure®) into the tube, where total glycans from the 10% or 20% ACN solutions have been completely dried in the previous step. The glycan solution in H₂O is mixed with an equal volume of NaCl solution, and the mixture of glycans and NaCl is loaded onto the DHB pre-spotted area of the Plate #2. The size of the area of the Plate #2 can be a circle of a 1700˜2000 μm diameter. Specifically, the diameter can be 1700 μm if the Plate #2 is a PFA2317DN0 plate supplied by HST®.

The step (e) is the drying process of the Plate #2 at the reduced pressure after the loading of glycan samples.

After purified glycans are loaded on all areas of the matrix pre-spotted Plate #2 as described in the above step (d), it is strongly recommended to evaporate solvents including water from the loaded sample by placing the Plate #2 in a reduced-pressure chamber. Glycans and NaCl are co-crystallized as compactly and homogenously as possible on the spots of the plate. The pressure of the drying chamber in this case can be around 6˜8×10⁻² torr, and where the temperature is not specified, it is assumed to be room temperature.

The fast drying process at a reduced pressure, in the present invention, is also considered to accelerate the rate of crystallization of the mixture of glycans and the matrix, and causes a reduction in the size of the crystals while facilitating even distribution of the crystals within the spot.

The step (f) is a step to obtain the MALDI-TOF mass spectra of the glycans from each area of the Plate #2.

MALDI-TOF mass spectrometric analysis can be executed on the mixed crystals of each spot of the Plate #2. The amount of each glycan sample and the matrix, the mixing ratio of the two solids on the spot, the selection of the kind of the matrix and its amount, and the polarity of the electromagnetic field can be properly chosen according to common knowledge of MALDI-TOF spectrometric technology.

The above glycans can be one or a combination of the two or more selected from the group of mannose, pentose, hexose, N-acetylglucosamine, N-acetylhexosamine, N-acetylneuraminic acid, hexosamine, sialic acid (including N-acetylneuraminic acid and N-glycolylneuraminic acid), and uronic acid (including GlaA), but it is not limited to one of the above glycans.

The analytical method of MALDI-TOF mass spectrometry, described in steps (a) to (f) in the present invention, can considerably improve reproducibility of the profiling of the glycans in the serum, and therefore can be applied to cancer screening or to differentiation of patients with benign tumors from those with borderline tumors. The above cancers are not specified, but can be ovarian cancer, breast cancer, lung cancer, bladder cancer, thyroid cancer, liver cancer (or hepatocellular carcinoma), diaphragm cancer, pancreatic cancer, cervical cancer, testicular cancer, colorectal cancer, anal cancer, bile duct cancer (or cholangiocarcinoma), gastric cancer (or stomach cancer), malignant tumors, esophageal cancer, gallbladder cancer, rectal cancer, appendix cancer, small intestine cancer, kidney cancer (or renal cancer), central nervous system cancers, skin cancer, choriocarcinoma, brain cancer, osteosarcoma, b-cell lymphoma, non-Hodgkin's lymphoma, Burkitt's lymphoma, fibrosarcoma, neuroblastoma, glioma, melanoma, monocytic leukemia, myelogenous leukemia, acute lymphocytic leukemia, or acute myelocytic leukemia.

In an exemplary embodiment, by using the analytical method of the glycans composed of the above steps (a) to (f), cancer patients can be screened out from healthy people on the basis of the serum-glycan analysis.

Therefore, the present invention offers the cancer screening method including the following steps:

(a) injecting each serum sample from healthy people and cancer patient into each area of the Plate #1 having a plurality of sample-loading areas;

(b) extracting at least one glycan from the sample by introducing glycan-releasing enzyme into each area of the Plate #1;

(c) preparing the Plate #2 having a plurality of sample-loading areas by spreading a matrix solution for MALDI-TOF mass spectrometry at least two times onto each area of the Plate #2 at 40 to 65° C., and subsequently drying and crystallizing the matrix;

(d) loading a solution including at least one glycan extracted from the step (b) onto each area of the Plate #2;

(e) drying the Plate #2 under reduced pressure; and

(f) obtaining a mass spectrum of at least one glycan from each area in the Plate #2 using MALDI-TOF.

Preferably, the above method can additionally include (g) a step to determine the cancer-specific glycans by comparing the mass spectra of the glycan(s) obtained from each area of the Plate #2. More specifically, by comparing the profiles of the peaks shown at each mass spectrum of cancer patients and healthy people, the peaks which show the variances of their intensities particularly in the cancer patients can be selected as the markers.

In another exemplary embodiment, by using the method of glycan analysis composed of the above steps (a) to (f), benign ovarian tumor patients can be differentiated from borderline ovarian tumor patients on the basis of the serum-glycan analysis.

Therefore, the present invention offers the method to differentiate benign ovarian tumor patients from the borderline ovarian tumor patients in the following steps:

(a) injecting each serum sample from benign ovarian tumor patients and borderline ovarian tumor patients into each area of the Plate #1 having a plurality of sample-loading areas;

(b) extracting at least one glycan from the sample by introducing glycan-releasing enzyme into each area of the Plate #1;

(c) preparing the Plate #2 having a plurality of sample-loading areas by spreading a matrix solution for MALDI-TOF mass spectrometry at least two times onto each area of the Plate #2 at 40 to 65° C., and subsequently drying and crystallizing the matrix;

(d) loading a solution including at least one glycan extracted from the step (b) onto each area of the Plate #2;

(e) drying the Plate #2 under reduced pressure; and

(f) obtaining a mass spectrum of at least one glycan from each area in the Plate #2 using MALDI-TOF.

As one good example, the above method can additionally include (g) the step to determine the tumor-specific glycans by comparing the mass spectra of the glycan(s) obtained from each area of the Plate #2. More specifically, the peak intensity of an identical glycan ion (identical mass-to-charge ratio) can be compared between the benign ovarian tumor patient group and the borderline ovarian tumor patient group. In other words, by comparing the mass spectra of serum glycans through pattern analysis, a significant difference in peak intensities of glycan mass spectra between different groups such as patients and healthy controls can provide clues to the possible candidate ion peaks to differentiate one group from the other.

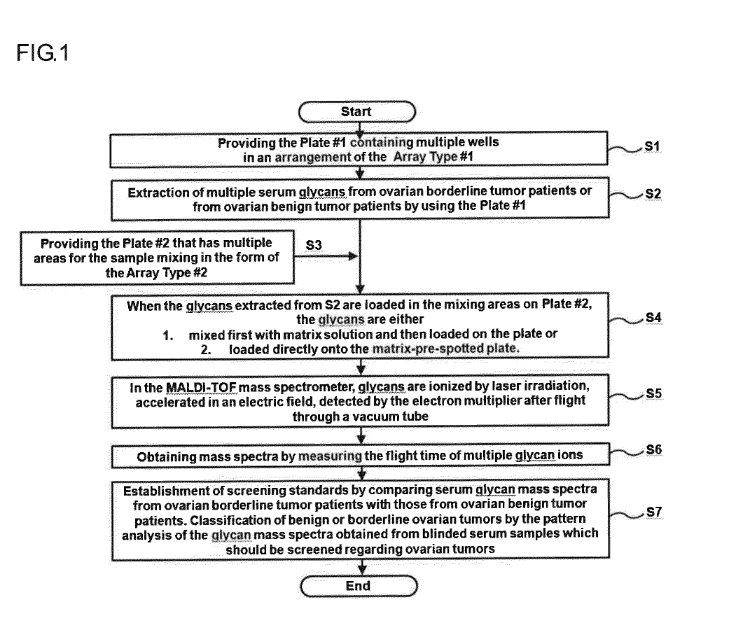

In the next step, the detection frequency of each peak and the intensity of the peak in each spectrum of a certain group of tumor patients are the important parameters of quantitative analysis for further investigation. The average values and the coefficients of variation of the intensity of each peak in the mass spectra can also be compared between different groups of patients to determine the key peaks as the markers to separate the groups of different tumors. By this analysis, the screening standards to differentiate benign ovarian tumor patients from borderline tumor patients can be established. This procedure corresponds to the step (S7) in FIG. 1 to establish the screening standards in the training groups. By this process, a specific group of the peaks are selected as the markers among many peaks in the mass spectra. Finally, the cut-off value of each selected marker to differentiate benign ovarian tumor patients from borderline ovarian patients is determined according to statistical analysis of the intensity variations of those marker peaks in the group of the same tumor patients.

If the intensity of a marker peak from the spectra of the borderline ovarian tumor patients' group and those of the benign ovarian tumor patients' group is compared with the marker's cut-off value established by previous analyses, it can determine the peak's likelihood to have resulted from either a borderline tumor patient or a benign tumor patient. All the marker peaks from a MALDI-TOF mass spectrum could be analyzed with respect to cut-off values of each peak, and if the tendency of each marker peak toward either tumor groups is summarized from a mass spectra, the total sum of the tendency, as a consequence, can determine whether the spectrum is speculated to belong to the benign ovarian tumor group or to the borderline ovarian tumor group.

The screening method for cancer patients or the differentiation method for tumor patients demonstrated in the present invention are based on the improved reproducibility and better accuracy of the newly introduced methodologies of MALDI-TOF mass spectrometry when compared to conventional procedures. The methods of the present invention present a much higher level of screening accuracy that the traditional ovarian tumor marker CA-125 has never reached before.

One example of the screening method of the present invention to differentiate the benign ovarian tumor patients from the borderline ovarian tumor patients can consist of the following steps: dividing each group of patients into two different subgroups, one of which is designated as a training set and the other as a blind test set; selecting one or more peaks as biomarkers for the discrimination between the benign ovarian tumor group and the borderline ovarian tumor group, in comprehensive statistical analyses comparing the intensities of ion peaks from the mass spectra between the two groups in the training set; establishing differentiation standards (cut-off values) for each biomarker peak on the basis of the analyses with mass spectra generated from the training set; analyzing the mass spectra from the blind test set with the procedures summarizing the likelihood of each marker peak toward one of the two groups as described previously, in order to independently validate the effectiveness of these screening standards determined by the training set of those two groups; and evaluating the competence of the screening methods based on the quantitative MALDI-TOF mass spectrometry of the N-linked serum glycans for the discrimination between benign ovarian tumor patients and borderline ovarian tumor patients.

According to specific examples of the invention, this analysis of serum glycans with highly reproducible MALDI-TOF mass spectrometry could lead to the discovery of new screening markers that are derived from serum glycans and have better specificity and sensitivity than those of conventional ovarian tumor marker CA-125 (cancer antigen 125). By using the Plate #1 having multiple wells with an arrayed pattern, 96 different samples were processed simultaneously. The enzyme reaction to release glycans from the serum proteins was rapidly completed within 10 minutes by continually applying microwave irradiation, while maintaining the reaction temperature constant with the cooling system using the air bubble effect. Otherwise, the reaction would have taken more than 12 hrs (overnight). Further, Plate #2 having multiple spot areas was used to load the glycans for MALDI-TOF mass spectrometry. The background surface as well as the individual areas for sample loading on the Plate #2 was fabricated so that the loaded material was highly condensed within each area. In addition to this surface fabrication, individual sample-loading areas of the Plate #2 were already coated with the matrix that had been sprayed multiple times upon a heating plate for fast drying that was required for very even distribution of the matrix material within each area. These features of the Plate #2, in combination with another rapid drying process for the loaded glycans under reduced pressure, drastically enhanced the homogeneity of co-crystals derived from the glycans and matrix. This uniform co-crystallization of the glycans with the matrix on the Plate #2 eventually led to minimized experimental errors (such as the coefficients of variation or relative standard deviation) in the MALDI-TOF mass spectrometry, and made it possible for the statistical analyses of the MALDI-TOF mass spectrometry data from serum glycans to accurately discriminate different ovarian tumor types. In summary, all the aspects of the present invention described above have streamlined the whole processes from blood to the mass spectrometry of the extracted glycans as one integrated batch procedure with high throughput and reproducibility.

By introducing several aspects described above to reduce any possible variance during the preparation processes of the serum samples and some improved techniques in the sample loading processes to minimize the data dispersion in the MALDI-TOF mass spectrometry, not only is the reproducibility of the data enhanced, but also the relative standard deviation (in other words, the coefficient of variation) were remarkably reduced.

More descriptions of the present invention by presenting some good examples as shown below are now listed for better understanding of the present invention, but these are for demonstration only, and do not limit the entire scope of the present invention at all.

### Example 1

### Glycan Analysis from Human Serum

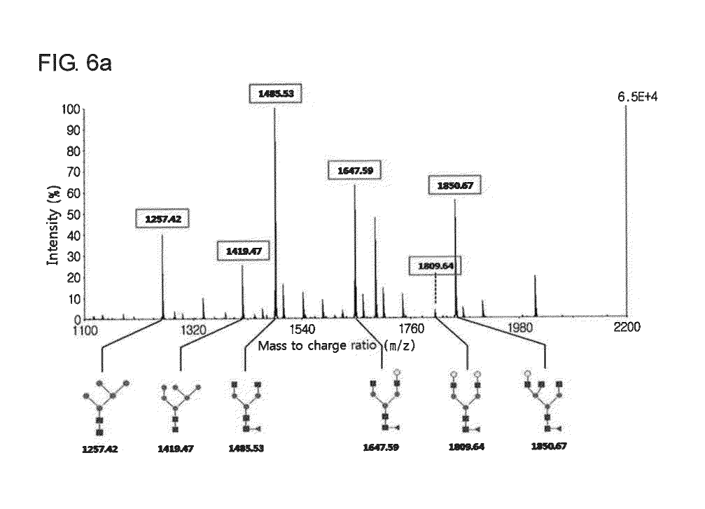

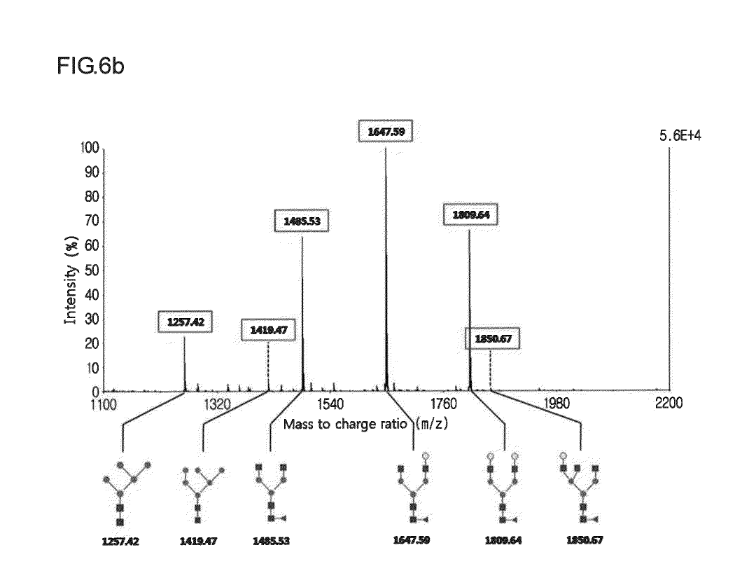

FIG. 6a and FIG. 6b show mass spectra of the glycans according to the method for the analysis of cancer-specific glycans in one example, originated from human sera of Sigma Aldrich. FIG. 6a shows a mass spectrum of multiple glycans finally eluted from a 10% ACN solution obtained in the positive mode, and FIG. 6b shows a mass spectrum of the multiple glycans finally eluted from a 20% ACN solution obtained in the same mode.

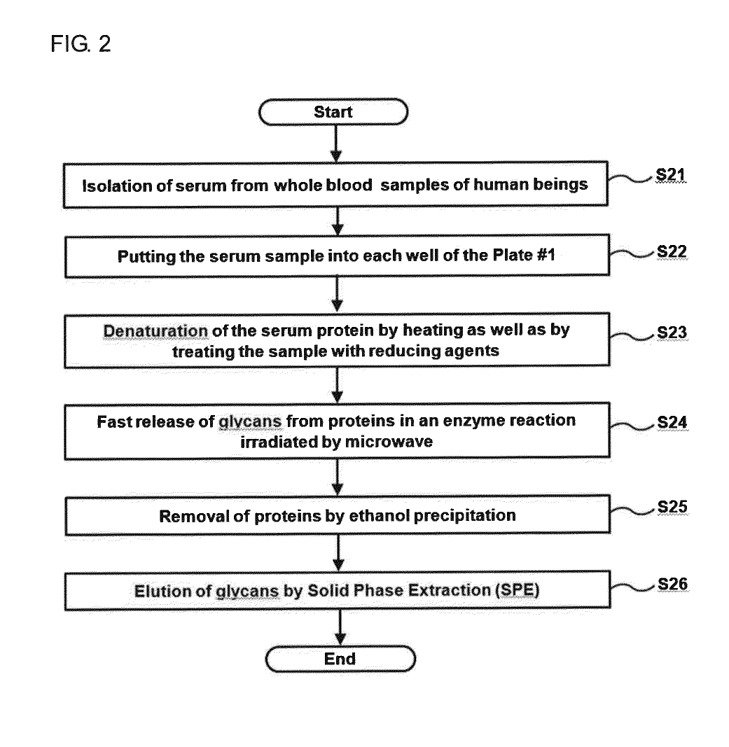

For further description, 50 μl of the human serum sample obtained from the process shown in FIG. 2, S22, was processed to the end of the step S26 in FIG. 2. After completion of the glycan extraction by the elution in a 10% or 20% ACN solution, each glycan in the tube is re-dissolved with 15 μl of Nanopure® water. Then 1 μl of the glycan sample is mixed with 1 μl of a NaCl solution (12 mM NaCl dissolved in 70% ACN) at a ratio of 1:1 (v/v) after which the mixture is laid on the DHB pre-spotted Plate #2. The DHB pre-spotted Plate #2 was prepared by spraying a 40 mg/ml DHB solution in ethanol 4 to 8 times to deposit around 1.5˜2.0 μg of the matrix crystal onto each spot of the plate. When the matrix solution was sprayed onto the exact spots of the plate, the plate was warmed at a temperature of around 40˜55° C. by a hot plate underneath. After all sample solutions were deposited onto each spot of the plate, respectively, the plate was dried in a vacuum chamber at a pressure of 6˜8×10⁻² torr. Subsequently, the Plate #2 was loaded into a MALDI-TOF mass spectrometer to gain the mass spectrum.

As demonstrated in the mass spectra, the distribution of the position of each glycan's mass to charge ratio (m/z) and its relative intensity were able to be identified around the molecular weight range from 1000 Da to 3000 Da. Also, the mass spectrum which shows the relative height of each ion peak was obtained.

### Example 2

### Glycan Analyses from Serum of Benign Ovarian Tumor Patients and Serum from the Borderline Ovarian Tumor Patients

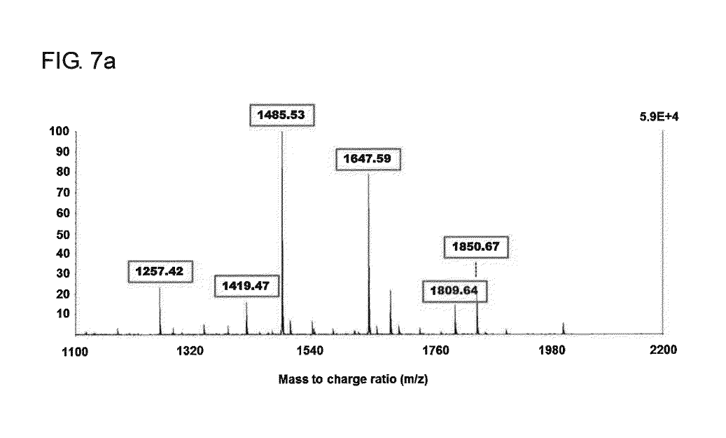

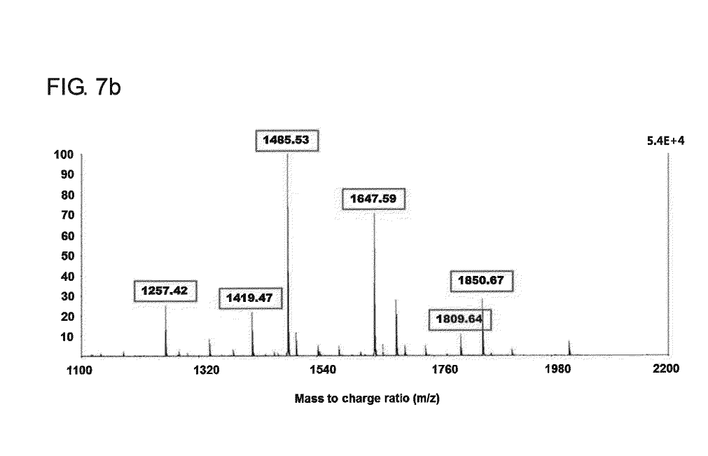

FIG. 7a shows one mass spectrum of the glycans of a 10% ACN fraction originated from a borderline ovarian tumor patient according to the method of cancer-specific glycan analysis in one example, and FIG. 7b shows a mass spectrum of the glycans from a 10% ACN fraction of a benign ovarian tumor patient. The experimental procedures are all the same as in Example 1. Consequently, as shown in the figures, the peak intensities of the glycans for the two patient groups at mass to charge ratios (m/z) of around 1257.42, 1419.47, 1485.53, 1647.59, 1809.64, and 1850.67 show relatively large differences. Therefore, these peaks with the m/z values, and thereby their corresponding glycans, could be selected as ovarian tumor markers.

TABLE 1 below shows the molecular structures and mass values identified by the method for the analysis of ovarian tumor-specific glycan biomarkers.

TABLE 1

| Ratio of mass to | |
|---|---|
| electric charge | |
| (m/z) | ^{()} Molecular Structure |
| 1257.42 | 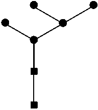 |
| 1419.47 | 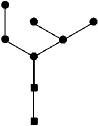 |
| 1485.53 | 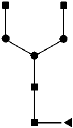 |
| 1647.59 | 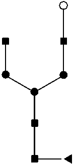 |
| 1809.64 | 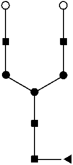 |
| 1850.67 | 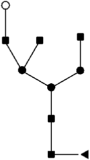 |

^{}

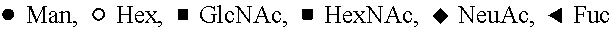

As shown in Table 1, the glycans selected as markers are mannose (Man, m/z 162.05); hexose (Hex, m/z 162.05); N-acetylglucosamine (GlcNAc, m/z 203.08); N-acetylhexosamine (HexNAc, m/z 203.08), N-acetylneuraminic acid (NeuAc, m/z 291.09), and fucose (Fuc, m/z 146.08), and any combination of the above glycans.

However, the above cancer markers are just some examples of the glycans in the serum which can be extracted and characterized by this invention. Therefore, they are not limited to as listed above. For example, oligosaccharide glycans used for profiling analysis of mass spectrometry shown in U.S. Pat. No. 7,651,847 can also be selected as cancer markers by the method for the analysis of ovarian cancer-specific or ovarian tumor-specific glycans according to the examples in the present invention. In addition, the peaks of other biomolecules detected by the MALDI-TOF mass spectrometry, which were separated and purified together with the glycans from human blood, can be included in the biomarkers identified by the present invention.

In the present invention, in order to enhance the efficiency of the mass spectrometry, the Plate #1 in the array format is used to prepare the samples quickly. Then the matrix pre-spotted Plate #2 is used to run the samples through MALDI-TOF MS. The present invention, therefore, provides a method of achieving high throughput and efficiency, which means a method capable of processing hundreds of samples simultaneously in one batch and in one integrated experimental procedure from the blood samples to the mass spectrometry. As a result, standardization and automation of the analysis procedures have been realized, which have rapidly brought clinically appropriate and reproducible results.

### Example 3

### Method of Glycan Analysis According to the Traditional Method and the Method Presented by the Present Invention

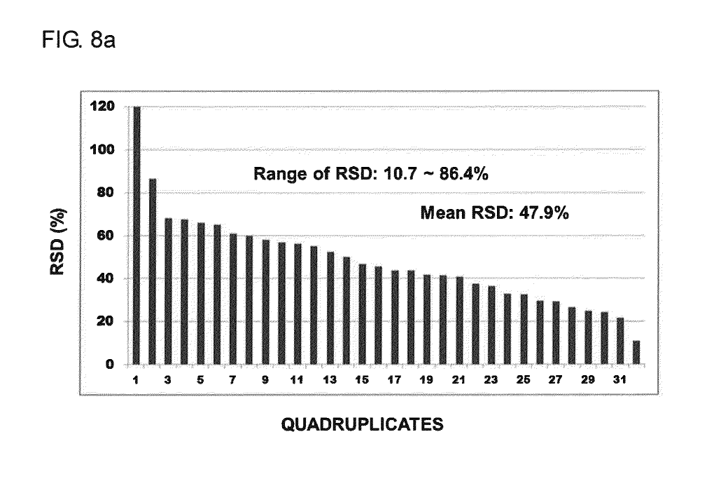

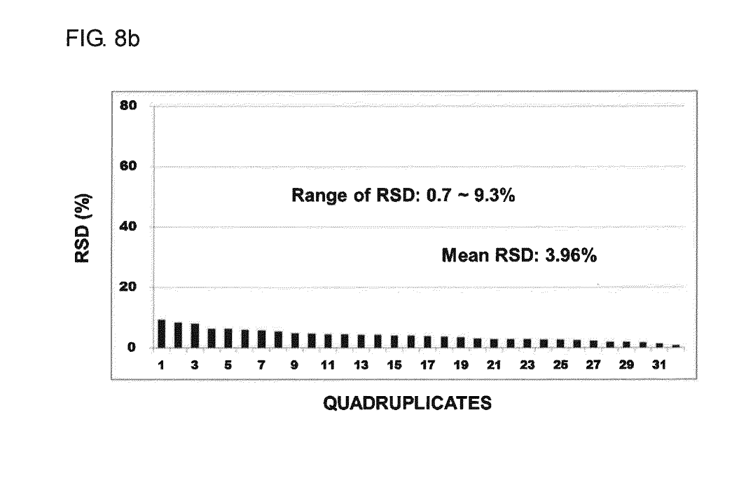

To confirm the reproducibility of the data, as an example, two methods were applied in parallel to a single human serum sample purchased from Sigma Aldrich. The same solution of 32 aliquots from one serum sample which was purified and finally eluted with a 10% ACN solution through the above whole procedures of glycan analysis by MALDI-TOF mass spectrometry was used. Firstly, as the traditional method, the matrix was mixed with the glycan, and then this mixture from each aliquot was applied on 4 different spots of a regular stainless steel plate not having self-focusing function, respectively, and then naturally dried before obtaining MALDI-TOF mass spectrometry. For a comparison purpose with the above procedure, the same glycan solution finally eluted in a 10% ACN solution was divided into identical 32 aliquots, and then MALDI-TOF mass spectra of the aliquots were obtained from 4 different spots of the Plate #2 for each aliquot. The glycan solution was respectively loaded on the DHB pre-spotted area and then the Plate #2 was immediately dried in a chamber at the reduced pressure before the MALDI-TOF analysis. FIGS. 8a and 8b show the bar graphs of the calculated coefficient of variation (CV; or RSD as relative standard deviation) of the absolute intensities obtained from the mass spectra of the same 32 aliquots. Four measurements of each aliquot were made from different areas, and all the intensities were measured at 1485.53 m/z at which the intensity was observed most distinctively in FIG. 6a, FIG. 7a, and FIG. 7b. In FIG. 8a, all values were calculated from the MALDI-TOF spectrum obtained from the traditional method of the sample loading process. The calculations in FIG. 8b were the same, but the MALDI-TOF spectra were obtained from DHB pre-spotted plate which was quickly dried under the reduced pressure immediately after the glycan sample loading.

The average value of the CV from the present invention was 3.96%, while that from the conventional method was 47.9% when compared in the above two graphs. This clearly shows that the reproducibility of the data obtained from MALDI-TOF mass spectrometry by the present invention has been improved by almost 10 times.

### Example 4

### Validation of Reproducibility of the Glycan Analysis of the Sera Derived from the Benign Ovarian Tumor Patients and from the Borderline Ovarian Tumor Patients

As an example, the below Table 2 presents the information of the age distribution of the patients of which serum samples were used for this example, including the sera of 169 patients with benign ovarian tumors and the sera of 68 patients with borderline ovarian tumors which were provided from the hospital.

TABLE 2
Study population

| | No. of | Mean age | Range |
|---|---|---|---|
| Tumor characteristics | patients | (±STDEV) | of age |
| Benign ovarian tumors | 169 | 34.4 ± 10.0 | 14-69 |
| Borderline ovarian tumors | 68 | 38.6 ± 14.2 | 19-83 |

The below Table 3 shows the mean values of the relative standard deviation of each peak in each MALDI-TOF mass spectrometry of the glycans extracted from each of 237 serum samples offered by the 237 individual patients. The mean RSD was calculated from two forms of the intensities: one from the absolute intensity of each peak of the spectrum, the other from the relative intensity of each peak normalized by the sum of all intensities shown in one spectrum. In addition, the two mean RSD values of the specific peak at 1485.53 m/z and the two RSDs of the peaks which appeared in every spectrum produced in this example are also listed in the following rows of the Table 3.

TABLE 3

| | Mean RSD | Mean RSD |
|---|---|---|
| | (absolute intensity) | (normalized intensity) |
| Mean of | 7.28% | 5.44% |
| all ion signals | | |
| m/z = 1485.53 | 5.74% | 1.91% |
| Ions with 100% | 8.36% | 4.37% |
| detection frequency | | |

All glycans extracted from each serum were deposited at 4 different spots in the DHB pre-spotted Plate #2, and those spots were independently used to obtain each MALDI-TOF mass spectrum. Therefore, the above mean RSDs were calculated from these quadruple measurements of the MALDI-TOF analysis. All the mean relative standard deviation values from 1.91% to 8.36% displayed in Table 3 are near the RSD value (3.96%) shown in FIG. 8b, which proves that the MALDI-TOF profiling analysis offered in the present invention has sufficient reproducibility and sensitivity to differentiate two different types of tumor patients: benign ovarian tumor and borderline ovarian tumor patients, of which symptoms are too similar to differentiate with classical methods.

### Example 5

### Confirmation of the Sensitivity and Specificity of the Glycan Analyses for Benign Ovarian Tumor Patients and Borderline Ovarian Tumor Patients

As an example, each group of benign ovarian tumor patients and borderline ovarian tumor patients, respectively, are randomly divided into two sub-groups; one group of a training set and the other group of a validation (or blind) set, respectively, in order to independently evaluate the efficiency of the differentiation power of the glycan profiling analysis by the MALDI-TOF mass spectrometry with the improved reproducibility and its application methods presented by the present invention. Those spectra obtained by the above “Glycan Profiling Analysis examined by the MALDI-TOF mass spectrometry with improved reproducibility and its application methods” were carefully examined and compared, then cut-off values for each marker peak were determined on the basis of intensity differences of each glycan peak between the two training groups to differentiate the benign tumors from the borderline tumors. Fifty patients of each group to compose the training set were randomly selected from 169 benign ovarian tumor patients and from 68 borderline ovarian tumor patients, respectively. The remained patients in each tumor type, therefore, 119 patients with benign ovarian tumors and 18 patients with borderline ovarian tumors belonged to the validation set. The accuracy of the screening standard made from the training set was actually validated by differentiation of these remaining patients in the blind set.

TABLE 4

| | | | Correct classification rate | | | |
|---|---|---|---|---|---|---|
| | Number of cases | | 1st cross-validation | | 2nd cross-validation | |
| | in the set | | Benign | Borderline | Benign | Borderline |
| Tumor type | Benign | Borderline | (n/total) | (n/total) | (n/total) | (n/total) |
| Training set | 50 | 50 | 68.0% (34/50) | 88.0% (44/50) | 64.0% (32/50) | 80.0% (40/50) |
| Test set | 119 | 18 | 66.4% (79/119) | 88.9% (16/18) | 69.7% (83/119) | 72.2% (13/18) |

| | | | | |
|---|---|---|---|---|
| Test set by CA-125 (cut-off, 35 U/ml) | 55.2% | 50.0% | 51.6% | 64.3% |

Table 4 summarizes the results from the cross validation of the training/blind sets of the patients as described above. As previously explained in Table 3, 72 peaks of 100% detection frequency shown in every MALDI-TOF mass spectrum of all patients in the training and validation groups were selected as the markers, and the cut-off values of each m/z peaks discriminating the benign tumor group from the borderline tumor group in the training set were determined by statistical analyses comparing the intensities of the 72 m/z peaks between the two groups of different ovarian tumor types.

In the 1ₛₜ cross validation, the specificity, which is the screening ability to determine the benign ovarian tumor patient to be the benign tumor patient, was 68.0%, and the sensitivity, which is the screening ability to determine the borderline ovarian tumor patient to be the borderline tumor patient, was 88.0%, as the results of internal evaluation in the training set. On the basis of this standard obtained from this training set, the specificity of the blind test set of the remaining 119 benign ovarian tumor patients was 66.4%, while the sensitivity of the blind test set of the remaining 18 borderline ovarian tumor patients was 88.9%. Meanwhile, the specificity and the sensitivity of CA-125 in the above blind test set were 55.2% and 50.0%, respectively, when the most conventional cut-off value of 35 IU/ml was used.

The 2^{nd} cross validation in Table 4 shows the result of one more similar types of evaluation. The only difference between the two evaluations was the composition of the patients in the training set and the blind test set. Therefore, the two evaluations are independent of each other.

As the summary of the above actual screening results, the screening power of CA-125 shows sensitivity of 50.0˜64.3% and specificity of 51.6˜55.2%, and the screening power of the N-linked glycan analysis method by MALDI-TOF shows sensitivity of 72.2˜88.9% and specificity of 64.0˜69.7%. Therefore, glycan analysis method shows better screening power than an immunoassay based on the antigen-antibody interaction such as the one using CA-125 for ovarian-related gynecological diseases. In case of sensitivity, the glycan analysis is better than CA-125 by at least 7.9% to a maximum of 38.9%, and in case of specificity, the glycan analysis is better than CA-125 by at least 8.8% to a maximum of 18.1%.

In conclusion, the differentiation method of the benign ovarian tumor patients from the borderline ovarian tumor patients by comparison of the 72 markers derived from the glycan analysis demonstrated superiority of the present methods to the screening method solely dependent on the CA-125, which counts on the concentration of the single serum antigen, mucin 16, at 23% higher sensitivity on average and 13% higher specificity on average, respectively.

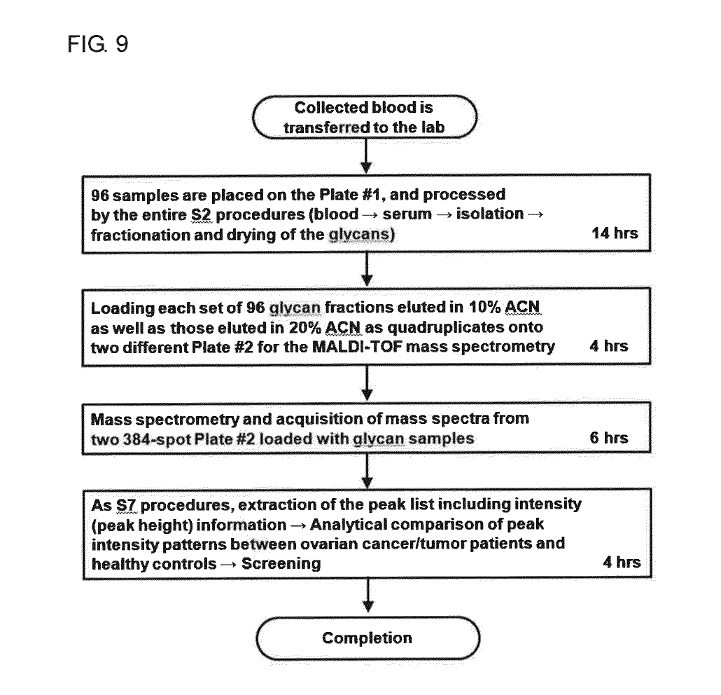

In the present invention as described above, new quantitative analytical methods with more efficiency and higher speed than any existing method or process has been established with the technique of each processing step from the human serum to the final extracted glycans. As schematically shown in FIG. 9, all preparative processes including the separation of sera from 96 individual samples of human blood by using the 96 wells arrayed in the Plate #1 as one batch, it was possible for the following MALDI-TOF mass spectrometry to obtain each spectrum of the glycans from each serum within 24 hours.

From each human blood sample, two fractions of glycans are purified by extraction with 10% and 20% ACN solutions, respectively, and then 8 spectra of MALDI-TOF analysis by quadruple replicates from each source of the glycans are obtained. Therefore, 768 spectra from one batch of 96 people (96×2×4) are provided, and the intensity values of each peak in a MALDI-TOF mass spectrum are collected and stored in the database. These numbers, thereafter, are compared by the pattern analysis in the reference sets of training groups, such as the groups of borderline ovarian tumor patients and benign ovarian tumor patients, or furthermore, the groups of ovarian cancer patients and healthy people. The pattern analysis of the peak intensities between different groups eventually results in the most accurate cut-off values for each marker peaks of the glycan MALDI-TOF mass spectrometry to differentiate one group from the other. With these established cut-off values, it is possible for newly examined test samples of 96 people to be screened for a specific disease within 4 hours once the MALDI-TOF mass spectra of glycans from the new samples have been collected.

In conclusion, the present invention has opened a new commercially available way to obtain results of screening tests with improved accuracy and high speed for 100 examinees within 48 hours.

The methods for the analysis of ovarian tumor-specific glycans in the examples were explained according to the reference procedures shown in FIG. 1 and FIG. 2 in the present invention. The above methods were presented and described in the flow charts for simple and conceptual explanation. Nevertheless, the present invention is not confined to these flow charts, which means that some processes in the blocks can be performed in different sequences or performed concurrently. Different routes, branches, or sequences of similar or the same procedures described in the flow charts shown in the FIG. 1 and FIG. 2 are also possible in order to reach similar or the same results. In addition, in some cases, not all the steps of the flow charts are necessarily required to follow the methods described in the present invention. Furthermore, the methods for the analysis of ovarian tumor-specific glycans and some parts of the screening procedures based on the statistical analyses can be realized as computer programs to operate the series of the procedures, and the computer programs can be recorded and retrieved as a magnetic recording system or optical recording system.

The present invention has been explained in all the examples in detail by using figures and tables. These examples are just representative for the clear understanding of the present invention, and it should be noted that those with common knowledge in this art will acknowledge that various modifications and applications on the basis of these examples are possible within the original scope and spirit of the present invention. These modifications, thus, cannot be excluded from the range of technical protection of the present invention. Therefore, the authentic range of technical protection of the present invention has to be defined on the basis of the technology platform attached as the patent-claimed items.

What is claimed is:

1. A method of glycan analysis comprising the steps of:
(a) injecting individual human serum samples into each area of a Plate #1 having a plurality of sample-loading areas;
(b) extracting at least one glycan from the sample by introducing glycan-releasing enzyme into each area of the Plate #1;
(c) preparing a Plate #2 having a plurality of sample-loading areas by spreading a matrix solution for MALDI-TOF mass spectrometry at least two times onto each area of the Plate #2 at 40 to 65° C., and subsequently drying and crystallizing the matrix;
(d) loading a solution comprising at least one glycan extracted from the step (b) onto each area of the Plate #2;
(e) drying the Plate #2 under reduced pressure; and
(f) obtaining a mass spectrum of at least one glycan from each area in the Plate #2 using MALDI-TOF,
wherein an average coefficient of variation of intensity of each obtained peak from the mass spectrometry is less than 10%.

2. The method of claim 1, wherein a number of sample-loading areas of the Plate #2 is equal to or more than a number of sample-loading areas of the Plate #1.

3. The method of claim 1, wherein the Plate #1 is a 6, 12, 24, 48, 96, or 384-well plate.

4. The method of claim 1, wherein the Plate #2 is a sample microfocusing plate for MALDI-TOF mass spectrometry, comprising at least two sample-loading areas comprising a central portion with a hydrophilic surface for condensation of a loaded material and a peripheral portion surrounding the central portion with a hydrophobic surface.

5. The method of claim 1, wherein the step (b) comprises the step of:
(b-1) denaturing proteins in the human serum sample by heating the Plate #1;
(b-2) treating the glycan-releasing enzyme with the sample to release glycans from the proteins in the sample; and
(b-3) separating at least one glycan from the proteins in the sample.

6. The method of claim 5, wherein the step (b-1) is performed by heating the Plate #1 after adding dithiothreitol, 2(β)-mercaptoethanol, or TCEP [tris(2-carboxyethyl)phosphine] into the sample to denature the proteins in the sample.

7. The method of claim 5, wherein the step (b-2) is performed in a water bath comprising a temperature controller, a microwave source radiating waves horizontally, and a system for generation of air bubbles.

8. The method of claim 7, wherein the step (b-2) is performed in the water bath where the sample is irradiated by microwaves directed horizontally in parallel to the surface of the water, while a reaction-containing lower portion of said Plate #1 is submerged in the water bath.

9. The method of claim 1, wherein the glycan-releasing enzyme in the step (b) is a peptide-N-glycosidase F.

10. The method of claim 1, wherein the matrix in the step (c) is 2,5-dihydroxybenzoic acid.

11. The method of claim 1, wherein the Plate #2 in the step (c) prepared by placing the Plate #2 on top of a heating plate at a temperature between 40 and 65° C. and then spreading the matrix solution at least two times onto each area of the Plate #2, and subsequently drying and crystallizing the matrix.

12. The method of claim 1, wherein the step (e) is performed under reduced pressure of 6˜8×10⁻² torr.

13. The method of claim 1, wherein the glycan is one selected from the group consisting of mannose, pentose, hexose, N-acetylglucosamine, N-acetylhexhoamine, N-acetylneuraminic acid, hexosamine, sialic acid including N-acetylneuraminic acid and N-glycolylneuraminic acid, uronic acid including GlaA, and a combination thereof.

14. The method of claim 1, wherein each human serum sample in the step (a) is from a plurality of cancer patients.

15. The method of claim 1, wherein the each human serum sample in the step (a) is from at least one healthy people and at least one cancer patient.

16. The method of claim 15, further comprising a step (g) of selecting a cancer-specific glycan by comparing each mass spectrum of the glycan extracted from each area in the Plate #2.

17. The method of claim 1, wherein the each human serum sample in the step (a) is from at least one benign ovarian tumor patient and at least one borderline ovarian tumor patient.

18. The method of claim 17, further comprising a step (g) of selecting a tumor-specific glycan by comparing each mass spectrum of the glycan extracted from each area in the Plate #2.

19. A method for screening cancer, comprising the steps of:
(a) injecting each serum sample from healthy people and cancer patients into each area of the Plate #1 having a plurality of sample-loading areas;
(b) extracting at least one glycan from the sample by introducing a glycan-releasing enzyme into each area of the Plate #1;
(c) preparing the Plate #2 having a plurality of sample-loading areas by spreading a matrix solution for MALDI-TOF mass spectrometry at least two times onto each area of the Plate #2 at 40 to 65° C., and subsequently drying and crystallizing the matrix;
(d) loading a solution comprising at least one glycan extracted from the step (b) onto each area of the Plate #2;
(e) drying the Plate #2 under reduced pressure; and
(f) obtaining a mass spectrum of at least one glycan from each area in the Plate #2 using MALDI-TOF.

20. A method for differentiating benign ovarian tumors from borderline ovarian tumors, comprising the steps of:
(a) injecting individual serum samples from benign ovarian tumor patients and borderline ovarian tumor patients into each area of a Plate #1 having a plurality of sample-loading areas;
(b) extracting at least one glycan from the sample by introducing a glycan-releasing enzyme into each area of the Plate #1;
(c) preparing a Plate #2 having a plurality of sample-loading areas by spreading a matrix solution for MALDI-TOF mass spectrometry at least two times onto each area of the Plate #2 at 40 to 65° C., and subsequently drying and crystallizing the matrix;
(d) loading a solution comprising at least one glycan extracted from the step (b) onto each area of the Plate #2;
(e) drying the Plate #2 under reduced pressure; and
(f) obtaining a mass spectrum of at least one glycan from each area in the Plate #2 using MALDI-TOF.
